(19) United States
(12) Patent Application Publication
(10) Pub. No.: US 2024/0018585 A1
(43) Pub. Date: Jan. 18, 2024

(54) METHODS, COMPOSITIONS, AND DEVICES FOR THE RAPID DETERMINATION OF FETAL SEX

(71) Applicant: Gateway Genomics, LLC, San Diego, CA (US)

(72) Inventor: Christopher Jacob, La Jolla, CA (US)

(21) Appl. No.: 18/257,092

(22) PCT Filed: Dec. 14, 2021
(86) PCT No.: PCT/US2021/063385

§ 371 Date: Jun. 12, 2023

Related U.S. Application Data
(60) Provisional application No. 63/125,395, filed on Dec. 15, 2020.

(51) Int. Cl. C12Q 1/6879; C12Q 1/6816

(52) U.S. Cl. CPC C12Q 1/6879; C12Q 1/6816; C12Q 1/6806

(57) ABSTRACT

The present disclosure provides methods, compositions, and devices for the rapid and direct detection of the sex of a fetus. The disclosure also provides methods, compositions, and devices for detecting fetal nucleic acids in biological samples (e.g., blood, cervical mucus, or urine).

### CROSS-REFERENCE TO RELATED APPLICATIONS

This application claims the benefit of priority of U.S. Provisional Patent Applications No. 63/125,395, filed Dec. 15, 2020, which is hereby expressly incorporated by reference in its entirety.

### FIELD OF THE DISCLOSURE

The present disclosure provides methods, compositions, and devices for the rapid and direct detection of the sex of a fetus. The disclosure also provides methods, compositions, and devices for detecting fetal nucleic acids in biological samples (e.g., blood, cervical mucus, or urine).

### BACKGROUND OF THE DISCLOSURE

There are approximately 4 million live births in the United States each year. More than two-thirds of expectant parents want to know the sex of their baby as early as practical into pregnancy. There are currently a limited number of options for learning the sex of a fetus available to expectant parents during the 40 weeks of the typical human gestational period.

Ultrasound imaging has been used safely for decades and is considered highly accurate for determining fetal sex at 18 to 20 weeks of gestation. Amniocentesis may be used for determining fetal sex with high accuracy between 15 to 18 weeks gestational age but carries a miscarriage risk and is not available to most women. More recently, non-invasive prenatal testing (NIPT) has been made available for high risk pregnancies and is highly accurate for determining fetal sex from maternal blood between 11- and 13-weeks gestational age, and on average 15 weeks (G. Allahbadia, (2015) J Obstet Gynaecol India. 65(3):141-145). All methods of NIPT require blood samples to be collected by a trained phlebotomist and processed at a specialized laboratory. Test results are generally provided a week or two after the blood sample was collected.

Thus, there is a need in the art for methods and kits useful for determining fetal sex the same day that the sample is collected, preferably within a few hours or less, and without the need for sample processing in a laboratory. Additionally, there is a need in the art for methods and compositions for the rapid and direct detection of fetal nucleic acids in biological samples. The present disclosure meets this need by providing highly accurate, noninvasive methods for the rapid determination of fetal sex. The present disclosure further provides novel methods, assays, kits, and compositions for detecting fetal nucleic acids and determining fetal sex in a biological sample.

### SUMMARY OF THE DISCLOSURE

The present disclosure provides methods of determining the sex of a fetus in a pregnant subject, comprising: obtaining a biological sample from the subject; and detecting fetal Y-chromosome nucleic acids in the sample, thereby determining the sex of the fetus. In some embodiments, the disclosure provides a method of determining the sex of a fetus in a pregnant subject, comprising: obtaining a biological sample from the subject; and detecting fetal Y-chromosome nucleic acids in the sample, thereby determining the sex of the fetus. In other embodiments, the disclosure provides a method for determining the sex of a fetus comprising: a) obtaining a biological sample from a subject who is pregnant or suspected of being pregnant comprising fetal nucleic acids; b) performing isothermal amplification on one or more target fetal nucleic acids in the sample to generate reporter molecules if the target nucleic acids are present in the sample; c) detecting the reporter molecules, wherein detection of the reporter molecules indicates the presence of target fetal nucleic acids in the sample; and d) determining the sex of the fetus based on detecting the presence of the target fetal nucleic acids in the sample. In still other embodiments, the disclosure provides a method comprising: a) obtaining a biological sample from a subject who is pregnant or suspected of being pregnant comprising fetal nucleic acids; b) performing isothermal amplification on one or more target fetal nucleic acids in the sample to generate reporter molecules if the target nucleic acids are present in the sample; and c) detecting the reporter molecules, wherein detection of the reporter molecules indicates the presence of target fetal nucleic acids in the sample.

In some embodiments, the disclosure provides a method for determining the sex of a fetus comprising: a) obtaining a biological sample from a subject who is pregnant or suspected of being pregnant comprising fetal nucleic acids; b) contacting the sample with a CRISPR/Cas effector protein, a guide RNA, and a labeled reporter DNA molecule, wherein the labeled reporter DNA molecule is cleaved if one or more target fetal nucleic acids are present in the sample; c) detecting a signal produced by the cleavage of the labeled reporter DNA molecule by the CRISPR/Cas effector protein, wherein detection of the signal indicates the presence of the target fetal nucleic acids in the sample; and d) determining the sex of the fetus based on detecting the presence of the target fetal nucleic acids in the sample. In some embodiments, the disclosure provides a method comprising: a) obtaining a biological sample from a subject who is pregnant or suspected of being pregnant comprising fetal nucleic acids; b) contacting the sample with a CRISPR/Cas effector protein, a guide RNA, and a labeled reporter DNA molecule, wherein the labeled reporter DNA molecule is cleaved if one or more target fetal nucleic acids are present in the sample; and c) detecting a signal produced by the cleavage of the labeled reporter DNA molecule by the CRISPR/Cas effector protein, wherein detection of the signal indicates the presence of the target fetal nucleic acids in the sample.

In some embodiments, the disclosure provides a method for determining the sex of a fetus comprising: a) obtaining a biological sample from a subject who is pregnant or suspected of being pregnant comprising fetal nucleic acids; b) performing isothermal amplification on one or more target fetal nucleic acids in the sample; c) contacting the sample with a CRISPR/Cas effector protein, a guide RNA, and a labeled reporter DNA molecule, wherein the labeled reporter DNA molecule is cleaved if one or more target fetal nucleic acids are present in the sample; d) detecting a signal produced by the cleavage of the labeled reporter DNA molecule by the CRISPR/Cas effector protein, wherein detection of the signal indicates the presence of the target fetal nucleic acids in the sample; and e) determining the sex of the fetus based on detecting the presence of the target fetal nucleic acids in the sample. In some embodiments, the disclosure provides a method comprising: a) obtaining a biological sample from a subject who is pregnant or suspected of being pregnant comprising fetal nucleic acids; b) performing isothermal amplification on one or more target fetal nucleic acids in the sample; c) contacting the sample with a CRISPR/Cas effector protein, a guide RNA, and a labeled reporter DNA molecule, wherein the labeled reporter DNA molecule is cleaved if one or more target fetal nucleic acids are present in the sample; and d) detecting a signal produced by the cleavage of the labeled reporter DNA molecule by the CRISPR/Cas effector protein, wherein detection of the signal indicates the presence of the target fetal nucleic acids in the sample.

In some embodiments, the methods of the disclosure further comprise determining the sex of a fetus based on the detection of one or more target fetal nucleic acid sequences on the Y-chromosome. In other embodiments, the target fetal nucleic acid is cell-free fetal DNA. In yet other embodiments, the target nucleic acid is a single copy target sequence or a multi-copy target sequence. In other embodiments, the multicopy target sequence is present on the Y-chromosome in more than 20 locations, 30 locations, 40 locations, 50 locations, 100 locations, or 1,000 locations. In certain embodiments, the sample is blood, plasma, serum, saliva, urine, and/or cervical mucus. In some embodiments, the methods of the disclosure further comprise determining an amount of the target fetal nucleic acid in the sample. In other embodiments, the detection of the reporter molecule is detected in less than 30 minutes. In yet other embodiments, the detection of the reporter molecule is detected in less than 60 minutes. In some embodiments, the detection is carried with an electrochemical chip, a graphene field-effect transistor, a nanopore sense, an SMR sensor, a nanoelectrokinetic chip, or a microarray. In other embodiments, the detection is completed using a smartphone.

In certain embodiments, the methods of the disclosure further comprise amplifying the target DNA in the sample by loop-mediated isothermal amplification (LAMP), helicase-dependent amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR), and/or isothermal multiple displacement amplification (IMDA). In some embodiments, the methods of the disclosure further comprise contacting the biological sample with a preservative composition. In other embodiments, the methods of the disclosure further comprise contacting the biological sample with a CRISPR composition. In still other embodiments, the methods of the disclosure further comprise contacting the biological sample with an amplification composition. In some embodiments, the methods of the disclosure further comprise contacting the biological sample with a protectant composition. In certain embodiments, the protectant composition comprises dextran, trehalose, and/or pullulan. In other embodiments, the CRISPR composition comprises a CRISPR/Cas effector protein, a guide RNA, and a labeled reporter DNA molecule. In yet other embodiments, the guide RNA comprises more than one crRNA. In some embodiments, the CRISPR/Cas effector protein is Cas9, Cas12a, Cas 14a/b, and/or Cas 13a/b. In still other embodiments, the preservative composition comprises an anti-coagulant (e.g., ethylenediaminetetraacetic acid (EDTA), ethylene glycol-bis(O-aminoethyl ether)-N,N,N′,N′-tetraacetic acid (EGTA), heparin), an antimicrobial (e.g., imidazolidinyl urea), a sugar, and/or an amino acid. In certain embodiments, the amplification composition comprises an isothermal polymerase, primers, and a probe.

In some embodiments, the methods of the disclosure further comprise enriching the sample for fetal nucleic acids. In certain embodiments, the enrichment is achieved by separating plasma from whole blood, by selectively capturing fetal nucleic acids from the biological sample, and/or by selectively degrading maternal nucleic acids in the biological sample. In some embodiments, the Y-chromosome nucleic acids are cell-free fetal nucleic acids or genomic fetal nucleic acids from a fetal cell. In some embodiments, the methods of the disclosure further comprise isolating, enriching, and/or concentrating the fetal nucleic acids.

In some embodiments, the disclosure provides device for detecting the presence of one or more target fetal nucleic acids in a biological sample, the device comprising: a lateral flow strip; a detection region on said lateral flow strip comprising a detectable particle or label; a fluid sample comprising a maternal biological sample comprising fetal nucleic acids; wherein said detection region provides a visual colorimetric signal indicating the presence of the target fetal nucleic acid in the fluid sample in less than two hours by capillary flow. In some embodiments, the device of the disclosure further comprises a CRISPR composition, a preservative composition, an amplification composition, and/or a protectant composition.

In some embodiments, the sex of the fetus is determined with at least 90% accuracy. In other embodiments, the gestational age of the fetus is between 4 weeks and 20 weeks. In other embodiments, the sample is blood, plasma, serum, saliva, urine, and/or cervical mucus. In certain embodiments, the sample volume is less than 1 ml. In other embodiments, the biological sample is processed within 1 hour, within 24 hours, or within 48 hours. In some embodiments, the preservative is an anti-coagulant (e.g., ethylenediaminetetraacetic acid (EDTA), ethylene glycol-bis(O-aminoethyl ether)-N,N,N′,N′-tetraacetic acid (EGTA), heparin), an antimicrobial (e.g., imidazolidinyl urea), a sugar, and/or an amino acid.

In some embodiments, the disclosure provides a kit for collecting a biological sample from a pregnant subject for determining fetal sex, the kit comprising a blood collection tube, a lancet or a device for obtaining venous or capillary blood from the subject, and instructions. In some embodiments, the kits of the disclosure further comprise a decontaminating agent. In some embodiments, the decontaminating agent is bleach, an alcohol wipe, chlorhexidine gluconate, hydrogen peroxide, and/or iodine. In other embodiments, the instructions provide for sample collection at gestational age of 2 weeks, 3 weeks, 4 weeks, 5 weeks, 6 weeks, 7 weeks, 8 weeks, 9 weeks, 10 weeks, 11 weeks, or 12 weeks. In some embodiments, the kits of the disclosure further comprise a CRISPR composition, a preservative composition, an amplification composition, and/or a protectant composition. In some embodiments, the kits of the disclosure further comprise a lateral flow strip.

The methods, compositions, devices, and kits of the disclosure provide optimal sensitivity, specificity, and accuracy for fetal sex determination. In some embodiments, the methods of the disclosure determine the sex of the fetus with at least 40%, 50%, 60%, 70%, 75%, 80%, 85%, 90%, 95%, 99%, or 100% specificity. In some aspects, the methods, compositions, devices, and kits of the disclosure determine the sex of the fetus with at least 40%, 50%, 60%, 70%, 75%, 80%, 85%, 90%, 95%, 99%, or 100% sensitivity. In yet other aspects, the methods, compositions, devices, and kits of the disclosure determine the sex of the fetus with at least 40%, 50%, 60%, 70%, 75%, 80%, 85%, 90%, 91%, 92%, 93%, 94%, 95%, 96%, 97%, 98%, 99%, or 100% accuracy.

In some embodiments, the false positive rate of the methods, compositions, devices, and kits of the disclosure is less than 1%, less than 2%, less than 3%, less than 4%, less than 5%, less than 6%, less than 7%, less than 8%, less than 9%, less than 10%, less than 20%, or less than 25%.

The performance of the methods, compositions, devices, and kits of the disclosure have been determined in multiple populations. In some embodiments, the performance of the methods, compositions, devices, and kits of the disclosure have been determined in a population of at least 10, 20, 30, 40, 50, 60, 70, 80, 90, 100, 150, 200, 300, 400, 500, and/or 1,000 or more pregnant subjects.

The methods, compositions, devices, and kits of the disclosure may be used at various gestations ages of pregnancy. In some embodiments, the gestational age of the fetus is 2 weeks, 3 weeks, 4 weeks, 5 weeks, 6 weeks, 7 weeks, 8 weeks, 9 weeks, l0 weeks, 11 weeks, 12 weeks, 20 weeks, or 40 weeks. In some embodiments, the gestational age of the fetus is 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days 42 days, 49 days, 56 days, 63 days, 70 days, 77 days, 84 days, 180 days, or 250 days.

The disclosure provides methods, compositions, devices, and kits for detecting Y-chromosome nucleic acids in a biological sample from a pregnant subject.

In other embodiments, the methods further comprise interpreting data generated when detecting the Y-chromosome DNA. In some embodiments, the interpreting is performed using a machine learning algorithm, a cycle-threshold (CT) algorithm, or artificial intelligence.

In some embodiments, the biological sample is contacted with a preservative. In some embodiments, the preservative is an anti-coagulant (e.g., ethylenediaminetetraacetic acid (EDTA), ethylene glycol-bis(β-aminoethyl ether)-N,N,N′,N′-tetraacetic acid (EGTA), heparin), an antimicrobial (e.g., imidazolidinyl urea), a sugar, and/or an amino acid. In other embodiments, the preservative is a solid, a liquid, and/or a gel.

Various types of biological samples can be used with the methods, compositions, and kits of the disclosure. In some embodiments, the sample is blood, plasma, serum, saliva, urine, and/or cervical mucus. In other embodiments, the sample is maternal blood, maternal plasma, or maternal serum. In yet other embodiments, the volume of the sample obtained from the subject is 10 ul to 10 ml. In some embodiments, the volume of the sample used to detect Y-chromosome DNA is a microvolume. In certain embodiments, the microvolume is about 1,000 ul, about 900 ul, about 800 ul, about 700 ul, about 600 ul, about 500 ul, about 400 ul, about 300 ul, about 200 ul, about 150 ul, about 100 ul, about 50 ul, about 25 ul, about 10 ul. The biological sample can be processed at any time after being collected from the subject. In some embodiments, the biological sample is processed within 1 hour, within 24 hours, or within 48 hours. In other embodiments, the biological sample is not processed for at least 6 hours, at least 8 hours, at least 10 hours, at least 12 hours, at least 1 day, at least 2 days, at least 3 days, at least 1 week, at least, 2 weeks, or at least 4 weeks.

The methods, devices, and kits of the disclosure may include instructions for decontaminating the site on the pregnant subject where the sample will be collected. In certain embodiments, the decontamination is performed by applying bleach to the site of collection, by applying an alcohol wipe to the site of collection, by treating the site of collection with ultra-violet light, by applying chlorhexidine gluconate, hydrogen peroxide, and/or iodine to the site of collection, by applying a brush (e.g., a nail brush) to the site of the collection.

The present disclosure further provides devices and kits for obtaining a biological sample from a pregnant subject. The devices and kits may comprise a blood collection tube, a lancet or a device useful for obtaining venous or capillary blood from the subject, a tourniquet, a bandage, an alcohol swab, a nail or skin brush, and instructions for using the kits. In some embodiments, the kits further comprise a decontaminating agent. In certain embodiments, the decontaminating agent is bleach, an alcohol wipe, chlorhexidine gluconate, hydrogen peroxide, and/or iodine. In other embodiments, the device for obtaining venous or capillary blood is a lancet (e.g., BD Microtainer contact-activated lancet), a syringe, and/or a push-button blood collection device (e.g., a TAP device, Seventh Sense Biosystems). In some embodiments, the biological sample is collected into a tube, onto a card, and/or a swab.

The present disclosure provides methods for detecting Y-chromosome DNA in biological samples from pregnant subjects. In some embodiments, a set of nucleic acid primers and/or probe are used to amplify and/or detect the Y-chromosome DNA in the sample. Primers and probes used in the methods of the disclosure may target one or more targets or target regions on the Y-chromosome (e.g., a gene on the Y-chromosome). In some embodiments, the target on the Y-chromosome is SRY, DYS, or DAZ. In some embodiments, the methods use one or more targets on the Y-chromosome to detect Y-chromosome DNA in the sample. In other embodiments, the target is a DNA sequence that is present in one or more locations on the Y-chromosome.

Kits of the disclosure include instructions for collecting the sample at various gestational ages. In some embodiments, the instructions provide for sample collection at gestational age of 2 weeks, 3 weeks, 4 weeks, 5 weeks, 6 weeks, 7 weeks, 8 weeks, 9 weeks, 10 weeks, 11 weeks, 12 weeks, 13 weeks, 14, week, 15 weeks, 16 weeks, 17 weeks, 18 weeks, 19 weeks, 20 weeks, 21 weeks, 22 weeks, 23 weeks, 24 weeks, 25 weeks, 30 weeks, 35 weeks, or 40 weeks. In some embodiments, the gestational age of the fetus is 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days 42 days, 49 days, 56 days, 63 days, 70 days, 77 days, 84 days, 100 days, 150 days, 200 days, or 250 days.

The methods, compositions, devices, and kits of the disclosure can be used to detect very small amounts of Y-chromosome DNA in a biological sample from a pregnant subject. In some embodiments, the methods of the disclosure can detect about 1 to 0.1 genomic equivalent of cffDNA in a sample, about 0.9 genomic equivalent of cffDNA in a sample, about 0.8 genomic equivalent of cffDNA in a sample, about 0.7 genomic equivalent of cffDNA in a sample, about 0.6 genomic equivalent of cffDNA in a sample, about 0.5 genomic equivalent of cffDNA in a sample, about 0.4 genomic equivalent of cffDNA in a sample, about 0.3 genomic equivalent of cffDNA in a sample, about 0.2 genomic equivalent of cffDNA in a sample, about 0.1 genomic equivalent of cffDNA in a sample. In some embodiments, the fetal fraction in the biological sample is about 4%, about 3%, about 2%, about 1% or less than 1%.

These and other embodiments of the present disclosure will readily occur to those of skill in the art in light of the disclosure herein, and all such embodiments are specifically contemplated.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description. The disclosure is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items. It must be noted that as used herein and in the appended claims, the singular forms “a,” “an,” and “the” include plural references unless context clearly dictates otherwise.

### DESCRIPTION OF THE DISCLOSURE

It is to be understood that the disclosure is not limited to the particular methodologies, protocols, cell lines, assays, and reagents described herein, as these may vary. It is also to be understood that the terminology used herein is intended to describe particular embodiments of the present disclosure, and is in no way intended to limit the scope of the present disclosure as set forth in the appended claims.

It must be noted that as used herein and in the appended claims, the singular forms “a,” “an,” and “the” include plural references unless context clearly dictates otherwise. Thus, for example, a reference to “a nucleic acid” includes a plurality of such nucleic acids, a reference to a “composition” is a reference to one or more compositions and to equivalents thereof known to those skilled in the art, and so forth.

Unless defined otherwise, all technical and scientific terms used herein have the same meanings as commonly understood by one of ordinary skill in the art to which this disclosure belongs as read in light of the present disclosure. Although any methods and materials similar or equivalent to those described herein can be used in the practice or testing of the present disclosure, the preferred methods, devices, and materials are now described. All publications cited herein are incorporated herein by reference in their entirety for the purpose of describing and disclosing the methodologies, reagents, and tools reported in the publications that might be used in connection with the disclosure. Nothing herein is to be construed as an admission that the disclosure is not entitled to antedate such disclosure by virtue of prior disclosure.

The practice of the present disclosure will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

The present disclosure provides methods and compositions for the rapid and direct detection of fetal nucleic acids in a biological sample and determination of fetal sex. In particular, the inventors have developed a rapid fetal sex test that detects the presence of fetal nucleic acids in a biological sample from a pregnant subject. The disclosure demonstrates that fetal nucleic acids present in the maternal samples (e.g., maternal blood) may be detected with a rapid test to determine fetal sex in a subject.

The disclosure provides methods for determining fetal sex in a pregnant subject. In some embodiments, the methods determine fetal sex in the subject with at least 99% accuracy.

The disclosure also provides compositions for use in the methods described herein. Such compositions may include compounds, primers, probes, preservatives, including anticoagulants, cell fixatives, protease inhibitors, phosphatase inhibitors, proteins, DNA or RNA preservatives.

The present disclosure further provides kits for collecting biological samples from pregnant subjects or for determining fetal sex in a subject. In these embodiments, the kits comprise a blood collection tube, a lancet or a device useful for obtaining venous or capillary blood from the subject, a tourniquet, a bandage, an alcohol swab, a nail or skin brush, and instructions for using the kits.

The section headings are used herein for organizational purposes only, and are not to be construed as in any way limiting the subject matter described herein.

### Biological Sample

The disclosure provides methods, compositions, and kits for determining the sex of a fetus in a pregnant subject. Generally, the methods of the disclosure involve the detection of Y-chromosome DNA in a biological sample obtained from a pregnant subject. A biological sample comprising fetal nucleic acids may be obtained from a pregnant subject. The biological sample obtained from the subject is typically blood, but can be any sample from bodily fluids, tissue or cells comprising the nucleic acids to be analyzed. The biological sample may include, but is not limited to, whole blood, serum, plasma, urine, a cervical swab, saliva, a buccal swab, and/or amniotic fluid.

In some embodiments, the biological sample of the disclosure can be obtained from blood. In some embodiments, about 0.01-10 mL of blood is obtained from a subject. In other embodiments, about 10-50 mL of blood is obtained from a subject. In some embodiments, a single drop of blood is collected from a subject. Blood can be obtained from any suitable area of the body, including an arm, a leg, a finger, or blood accessible through a central venous catheter. In some embodiments, blood is collected from the finger using a lancet. In other embodiments, blood is collected from the arm via venipuncture. In yet other embodiments, blood is collected from the arm using a blood collection device (e.g., a TAP blood collection device, Seventh Sense Biosystems, MA). In some embodiments, blood is collected following a treatment or activity. For example, blood can be collected following a medical exam. The timing of collection can also be coordinated to increase the amount of fetal nucleic acids present in the sample. For example, blood can be collected following exercise or drinking orange juice.

Blood may be combined with various components following collection to preserve or prepare samples for subsequent testing. For example, in some embodiments, blood is treated with an anticoagulant, a cell fixative, a protease inhibitor, a phosphatase inhibitor, a protein, a DNA, or an RNA preservative following collection. In some embodiments, the biological sample is incubated with a buffer composition. In some embodiments, the biological sample is incubated with a cell stabilizer composition. In some embodiments, the biological sample is incubated with a preservative composition. In some embodiments, the preservative is an anti-coagulant (e.g., ethylenediaminetetraacetic acid (EDTA), ethylene glycol-bis(O-aminoethyl ether)-N,N,N′,N′-tetraacetic acid (EGTA), heparin), an antimicrobial (e.g., imidazolidinyl urea), a sugar, and/or an amino acid. In other embodiments, the preservative is a solid, a liquid, and/or a gel. In some embodiments, blood is collected via venipuncture using vacuum collection tubes containing an anticoagulant such as EDTA, EGTA, or heparin. Blood can also be collected using a heparin-coated syringe and hypodermic needle. Blood can also be combined with components that will be useful for subsequent analysis of the fetal nucleic acids contained therein.

The volume of the biological sample obtained from the subject may be 10 ul to 10 ml. In some embodiments, the volume of the sample used to detect Y-chromosome DNA is a microvolume. In certain embodiments, the microvolume is about 1,000 ul, about 900 ul, about 800 ul, about 700 ul, about 600 ul, about 500 ul, about 400 ul, about 300 ul, about 200 ul, about 150 ul, about 100 ul, about 50 ul, about 25 ul, about 10 ul. Blood samples are typically processed within a few hours from the time of collection to prevent significant degradation of the nucleic acids by enzymes present in blood. The methods of the disclosure enable the biological sample to be processed up to several months after being collected from the subject. In some embodiments, the biological sample is processed within 1 second, within 30 seconds, within 1 minute, within 10 minutes, within 30 minutes, within 1 hour, within 2 hours, within 12 hours, within 24 hours, or within 48 hours.

The biological sample should be free of contaminating DNA from a non-maternal or non-fetal source (e.g., touch DNA from another person). In various methods of the disclosure, the presence or absence of Y-chromosome DNA in the biological sample is used to determine if a fetus is male or female. Contaminating Y-chromosome DNA (i.e., non-fetal Y-chromosome DNA) has the potential to produce a false positive result for a fetal sex assay of the disclosure. In some aspects, maternal blood is collected from a site on the body which is generally free of contaminating Y-chromosome DNA. In some embodiments, the site of blood collection is the upper arm. A TAP blood specimen collection device is used to collect a maternal blood sample. The TAP Blood Collection Device is a single-use, sterilized blood collection and transportation device that uses a combination of two mechanisms, capillary action and vacuum extraction. The device consists of an integrated reservoir with a visual fill indicator window. The device is designed to collect and contain approximately 100-500 μL of capillary whole blood. The internal fluid path is coated with lithium heparin, EDTA, EGTA, or other anticoagulants and/or preservatives. The top of the device includes a button and a fill indicator window. The base of the device includes a release liner that covers a layer of hydrogel adhesive. The hydrogel adhesive seals to the skin and holds the device in place during use. The TAP device contains an array of microneedles in order to puncture through the skin. The microneedles are activated by a spring, released by pushing a button or lever on the device. The device is provided sterile in a tray and foil pouch. A preservative or cell stabilizer can optionally be used in the TAP device to stabilize cells and preserve nucleic acids. In some embodiment, the preservative in the TAP device prevents significant genomic DNA contamination in blood samples during the testing process. In some embodiments, the TAP device including the preservative, substantially prevents cell lysis and/or cell-free nucleic acid degradation of the blood sample due to DNase and RNase activity after blood collection during the testing process.

In some embodiments, a blood sample is further processed to separate the plasma fraction from the cellular fraction of the blood. A separation method that utilizes immunological capture and filtration to exclude cells from plasma may be used (Su et al. Micromachines 2020, 11, 352). In this method, the red blood cells can be captured and immobilized by antibody coated in separation matrix, and residue cells can be totally removed from the sample by a commercially plasma purification membranes. A 400 uL anti-coagulated whole blood sample with 65% hematocrit (Hct) can be separated by the device in 5 min with only one pipette. Up to 97% of the plasma can be recovered from the raw blood sample with a separation efficiency at 100%. In another method, a highly asymmetric membrane is used for the generation of plasma from whole blood. The highly asymmetric nature of the membrane allows the cellular components of blood (red cells, white cells, and platelets) to be captured in the larger pores without lysis, while the plasma flows down into the smaller pores on the downstream side of the membrane (e.g., VIVID plasma separator membrane, PALL). The rapid separation process yields plasma similar in HPLC and SDS PAGE profiles to traditional centrifuged plasma in less than two minutes.

### Pregnant Subjects

The disclosure provides methods, compositions, and kits for the determination of the sex of a fetus in a pregnant subject. The pregnancy may be the result of natural conception (i.e., a natural pregnancy) of result from use of assisted reproductive technology (e.g., in-vitro fertilization). In some embodiments, the pregnant subject has used assisted reproductive technology (ART) to become pregnant. In some aspects, the assisted reproductive technology is in-vitro fertilization, use of fertility medication (e.g., clomifene), ovulation induction, cryopreservation, and/or intracytoplasmic sperm injection. In some embodiments, the pregnant subject has a high-risk pregnancy. In other embodiments, the pregnant subject is a carrier of a sex-linked recessive disease or disorder.

The disclosure provides methods, compositions, and kits useful for determining fetal sex at various timepoints in pregnancy. Gestational age is a measure of the age of a pregnancy which is taken from the beginning of the woman's last menstrual period (LMP), or the corresponding age of the gestation as estimated by a more accurate method if available. Such methods include adding 14 days to a known duration since fertilization (as is possible in in vitro fertilization), or by obstetric ultrasonography. In some embodiments, the gestational age of the fetus is 2 weeks, 3 weeks, 4 weeks, 5 weeks, 6 weeks, 7 weeks, 8 weeks, 9 weeks, 10 weeks, 11 weeks, 12 weeks, 13 weeks, 14, week, 15 weeks, 16 weeks, 17 weeks, 18 weeks, 19 weeks, 20 weeks, 21 weeks, 22 weeks, 23 weeks, 24 weeks, 25 weeks, 30 weeks, 35 weeks, or 40 weeks. In some embodiments, the gestational age of the fetus is 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days 42 days, 49 days, 56 days, 63 days, 70 days, 77 days, 84 days, 100 days, 150 days, 200 days, or 250 days.

### Test Performance

The methods, compositions, and kits of the disclosure may be used in tests to determine fetal sex in a pregnant subject. Fetal sex test performance can be assessed by determining the test's sensitivity, specificity, area under the ROC curve (AUC), accuracy, positive predictive value (PPV), and negative predictive value (NPV). Disclosed herein are tests for determining fetal sex in a pregnant subject.

The performance of the tes may be based on sensitivity. The sensitivity of a test of the disclosure may be at least about 40%, 45%, 50%, 55%, 60%, 65%, 70%, 75%, 80%, 85%, 90%, 91%, 92%, 93%, 94% 95%, 96%, 97%, 98%, 99%, or 100%. The performance of the test may be based on specificity. The specificity of a test of the disclosure may be at least about 40%, 45%, 50%, 55%, 60%, 65%, 70%, 75%, 80%, 85%, 90%, 91%, 92%, 93%, 94% 95%, 96%, 97%, 98%, 99%, or 100%. The performance of the test may be based on area under the ROC curve (AUC). The AUC of a test of the disclosure may be at least about 0.5, 0.55, 0.6, 0.65, 0.7, 0.75, 0.8, 0.85, 0.9, or 0.95. The performance of the test may be based on accuracy. The accuracy of a test of the present disclosure may be at least about 40%, 45%, 50%, 55%, 60%, 65%, 70%, 75%, 80%, 85%, 90%, 91%, 92%, 93%, 94% 95%, 96%, 97%, 98%, 99%, or 100%.

The performance of the methods, compositions, and kits of the disclosure have been determined in multiple populations. In some embodiments, the performance of the methods, compositions, and kits of the disclosure have been determined in a population of at least 10, 20, 30, 40, 50, 60, 70, 80, 90, 100, 150, 200, 300, 400, 500, and/or 1,000 or more pregnant subjects. In certain aspects the accuracy of a test of the disclosure is determined in a population of at least 10, 20, 30, 40, 50, 60, 70, 80, 90, 100, 150, 200, 300, 400, 500, and/or 1,000 or more pregnant subjects. In certain aspects the sensitivity of a test of the disclosure is determined in a population of at least 10, 20, 30, 40, 50, 60, 70, 80, 90, 100, 150, 200, 300, 400, 500, and/or 1,000 or more pregnant subjects. In certain aspects the specificity of a test of the disclosure is determined in a population of at least 10, 20, 30, 40, 50, 60, 70, 80, 90, 100, 150, 200, 300, 400, 500, and/or 1,000 or more pregnant subjects.

### Optional Amplification of Nucleic Acids in the Sample

The disclosure provides methods for amplifying nucleic acids from a biological sample. In some embodiments, the sensitivity of a test of the disclosure can be increased by coupling detection with nucleic acid amplification. In some embodiments, the nucleic acids in a sample are amplified prior to contact with CRISPR compositions. In other embodiments, the nucleic acids in a sample are amplified simultaneous with contact with CRISPR compositions. For example, in some embodiments, a test includes amplifying nucleic acids of a sample (e.g., by contacting the sample with amplification compositions) prior to contacting the amplified sample with CRISPR compositions. In some embodiments, a test includes contacting a sample with amplification compositions at the same time (simultaneous with) that the sample is contacted with CRISPR compositions.

In some embodiments, the nucleic acids are amplified (e.g., by contact with amplification compositions) prior to contacting the amplified nucleic acids with CRISPR compositions. In some cases, amplification occurs for 10 seconds or more, (e.g., 30 seconds or more, 45 seconds or more, 1 minute or more, 2 minutes or more, 3 minutes or more, 4 minutes or more, 5 minutes or more, 7.5 minutes or more, 10 minutes or more, etc.) prior to contact with CRISPR compositions. In some cases, amplification occurs for 2 minutes or more (e.g., 3 minutes or more, 4 minutes or more, 5 minutes or more, 7.5 minutes or more, 10 minutes or more, etc.) prior to contact with CRISPR compositions. In some cases, amplification occurs for a period of time in a range of from 10 seconds to 60 minutes (e.g., 10 seconds to 40 minutes, 10 seconds to 30 minutes, 10 seconds to 20 minutes, 10 seconds to 15 minutes, 10 seconds to 10 minutes, 10 seconds to 5 minutes, 30 seconds to 40 minutes, 30 seconds to 30 minutes, 30 seconds to 20 minutes, 30 seconds to 15 minutes, 30 seconds to 10 minutes, 30 seconds to 5 minutes, 1 minute to 40 minutes, 1 minute to 30 minutes, 1 minute to 20 minutes, 1 minute to 15 minutes, 1 minute to 10 minutes, 1 minute to 5 minutes, 2 minutes to 40 minutes, 2 minutes to 30 minutes, 2 minutes to 20 minutes, 2 minutes to 15 minutes, 2 minutes to 10 minutes, 2 minutes to 5 minutes, 5 minutes to 40 minutes, 5 minutes to 30 minutes, 5 minutes to 20 minutes, 5 minutes to 15 minutes, or 5 minutes to 10 minutes). In some cases, amplification occurs for a period of time in a range of from 5 minutes to 15 minutes. In some cases, amplification occurs for a period of time in a range of from 7 minutes to 12 minutes.

In some embodiments, a sample is contacted with amplification compositions at the same time as contact with CRISPR compositions. In some embodiments, the CRISPR compositions are inactive at the time of contact and are activated once nucleic acids in the sample have been amplified.

In some embodiments, the amplification is isothermal amplification. The term “isothermal amplification” indicates a method of nucleic acid (e.g., DNA) amplification (e.g., using enzymatic chain reaction) that can use a single temperature incubation thereby obviating the need for a thermal cycler. Isothermal amplification is a form of nucleic acid amplification which does not rely on the thermal denaturation of the target nucleic acid during the amplification reaction and hence may not require multiple rapid changes in temperature. Isothermal nucleic acid amplification methods can therefore be carried out inside or outside of a laboratory environment. By combining with a reverse transcription step, these amplification methods can be used to isothermally amplify RNA.

Examples of isothermal amplification methods include but are not limited to: loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA).

In some embodiments, the amplification is recombinase polymerase amplification (RPA) (see, e.g., U.S. Pat. Nos. 8,030,000; 8,426,134; 8,945,845; 9,309,502; and 9,663,820, which are hereby incorporated by reference in their entirety). Recombinase polymerase amplification (RPA) uses two opposing primers (much like PCR) and employs three enzymes—a recombinase, a single-stranded DNA-binding protein (SSB) and a strand-displacing polymerase. The recombinase pairs oligonucleotide primers with homologous sequence in duplex DNA, SSB binds to displaced strands of DNA to prevent the primers from being displaced, and the strand displacing polymerase begins DNA synthesis where the primer has bound to the target DNA. Adding a reverse transcriptase enzyme to an RPA reaction can facilitate detection RNA as well as DNA, without the need for a separate step to produce cDNA. One example of components for an RPA reaction is as follows (see, e.g., U.S. Pat. Nos. 8,030,000; 8,426,134; 8,945,845; 9,309,502; 9,663,820): 50 mM Tris pH 8.4, 80 mM Potassium actetate, 10 mM Magnesium acetate, 2 mM DTT, 5% PEG compound (Carbowax-20M), 3 mM ATP, 30 mM Phosphocreatine, 100 ng/μl creatine kinase, 420 ng/μl gp32, 140 ng/μl UvsX, 35 ng/μl UvsY, 2000M dNTPs, 300 nM each oligonucleotide, 35 ng/μl Bsu polymerase, and a nucleic acid-containing sample).

In some embodiments, the amplification is loop mediated amplification (LAMP). LAMP employs a thermostable polymerase with strand displacement capabilities and a set of four or more specific designed primers. Each primer is designed to have hairpin ends that, once displaced, snap into a hairpin to facilitate self-priming and further polymerase extension. In a LAMP reaction, though the reaction proceeds under isothermal conditions, an initial heat denaturation step is required for double-stranded targets. In addition, amplification yields a ladder pattern of various length products. In some embodiments, the amplification is strand displacement amplification (SDA). SDA combines the ability of a restriction endonuclease to nick the unmodified strand of its target DNA and an exonuclease-deficient DNA polymerase to extend the 3′ end at the nick and displace the downstream DNA strand.

### Nucleic Acids

The disclosure provides methods, compositions, tests, and kits for detecting nucleic acids in a biological sample obtained from a pregnant subject. In some embodiments, the nucleic acids are cell-free fetal nucleic acids (e.g., cffDNA). In some embodiments, the nucleic acids are genomic fetal nucleic acids from a fetal cell (e.g., gfDNA). In other embodiments, the nucleic acids are fetal RNA. In some embodiments, the DNA and/or RNA is methylated DNA and/or methylated RNA. In some embodiments, the sequence of the nucleic acids of the disclosure may range in length from about 15 nucleotides to the full length of the sequence on the Y-chromosome. In one embodiment of the disclosure, the nucleic acid sequences are at least about 15 nucleotides in length. In another embodiment, the nucleic acid sequences are at least about 20 nucleotides in length. In a further embodiment, the nucleic acid sequences are at least about 25 nucleotides in length. In another embodiment, the nucleic acid sequences are between about 15 nucleotides and about 500 nucleotides in length. In other embodiments, the nucleic acid target sequences are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the nucleic acid sequences are at least 15 nucleotides in length. In some embodiments, the nucleic acid sequences are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

In one embodiment, the nucleic acid detected by a device of the present disclosure is one or more target sequences selected from the Y-chromosome. In some embodiments, the target sequences are present on the Y-chromosome in multiple locations. In certain embodiments, the target sequences are present in 2, 3, 4, 5, 6, 7, 8, 9, 10, about 15, about 20, about 25, about 50, about 75, about 100, about 150, about 200, about 300, about 400, about 500, about 750, about 1,000 locations on the Y-chromosome. The sensitivity of assays of the disclosure can be increased by detecting and/or amplifying target sequences that are present on the Y-chromosome in multiple locations. A label can be attached to or incorporated into a probe or primer polynucleotide to allow detection and/or quantitation of a target nucleic acid representing the target sequence of interest.

The detection of a plurality of target sequences on the Y-chromosome may be carried out separately or simultaneously with one test sample. In some embodiments, the target nucleic acid on the Y-chromosome is SRY, DYS, and/or DAZ. An assay consisting of a combination of the target sequences referenced in the instant disclosure may be constructed. Such a panel may be constructed using 1, 2, 3, 4, 5, 6, 7, 8, 9, 10, 15, 20, or more or target sequences. The detection of a single target sequence or subsets of target sequences comprising a larger panel of Y-chromosome targets could be carried out with the methods described within the instant disclosure to optimize assay sensitivity or specificity in various settings. The ratio of a target sequence on the Y-chromosome and a control sequence from an autosomal chromosome may be used for determining fetal sex.

### CRISPR/Cas Compositions

CRISPR/Cas systems, compositions and chemistry may be used in the methods of the present disclosure to detect the presence of a nucleic acid in the biological sample. CRISPR/Cas systems can be divided into two classes, each consisting of several types and subtypes (Makarova et al., 2020 which is hereby incorporated by reference in its entirety). CRISPR/Cas class 1 systems comprise complexes of multiple effector proteins, where each protein performs a single function in the CRISPR process. Class 2 CRISPR/Cas systems are characterized by one single effector protein that has multiple functions within the CRISPR process. Class 2 systems are most used in bioengineering and CRISPR diagnostics due to their simplicity in combination with their high efficiency. Class 2 systems can be subdivided in 3 types commonly used in CRISPR sensing: Type II, V and VI.

Class 2 CRISPR/Cas systems are characterized by a single multidomain protein that associates with an RNA sequence to form a ribonucleoprotein (RNP) surveillance complex. This RNA sequence is called guide RNA (gRNA). gRNA consists of a customizable component called the crRNA, that defines the specificity and selectivity towards target DNA, and a non-coding RNA part containing two-dimensional structures. This non-coding part facilitates association between gRNA and the effector protein by extensive hydrogen bond contacts and aromatic stacking between gRNA and the effector protein (Chen and Doudna, 2017; Slaymaker et al., 2019, which are hereby incorporated by reference in their entirety). The interaction between the gRNA and the protein induces structural changes to the effector protein, as confirmed by crystallography (Nishimasu et al., 2014; Slaymaker et al., 2019; Yamano et al., 2016, which are hereby incorporated by reference in their entirety). These structural changes ‘activate’ the effector protein and induce the formation of the RNP surveillance complex, which scans nucleic acids and targets sequences complementary to its crRNA for enzymatic degradation.

One or more cnRNA can be used in the methods of the disclosure. In some embodiments, a single crRNA, two crRNAs, three crRNAs, four crRNAs, five crRNAs, six crRNAs, seven crRNAs, eight crRNAs, nine crRNAs, ten crRNAs, or more than 10 crRNAs are used in the methods of the disclosure.

### Shelf-Stable Protective Compositions

Shelf-stable protective compositions are provided by the present disclosure. In some embodiments, the compositions are reagents used to amplify and/or detect the nucleic acids are room temperature stable. Such protective compositions serve to enhance the shelf-life of the other compositions used in the present disclosure. In some embodiments, the protectant comprises 2% trehalose, 10% pullulan. In some embodiments, the 2% trehalose, 10% pullulan is mixed with CRISPR compositions in a volume ratio of 1:1. In certain embodiments, the mixture is dried with heating into a small film sheet. In other embodiments, the CRISPR compositions are freeze dried and/or lyophilized. In other embodiments, the protective reagents comprise trehalose and dextran. In some embodiments, the protective reagents comprise about 10% trehalose, about 20% trehalose, about 30% trehalose, about 40% trehalose, about 50% trehalose, about 80% trehalose, or about 90% trehalose. In other embodiments, the protective reagents comprise about 10% dextran, about 20% dextran, about 30% dextran, about 40% dextran, about 50% dextran, about 80% dextran, or about 90% dextran.

In some embodiments, the protective compositions further comprise amplification compositions and/or detection compositions. In some embodiments, the protective compositions are present in a sample container prior to sample collection. In other embodiments, the protective compositions are added to the biological sample after sample collection. In some embodiments, the detection reagents are deposited onto the lid of the container used to collect the biological sample.

### Detection Methods and Devices

The present disclosure provides methods for the rapid detection of fetal nucleic acids in biological sample. Multiple methods of detection may be used including, for example, fluorescence, colorimetric, or electronic methods of detection.

Electronic methods of detection specifically contemplated include: an electrochemical chip; a graphene filed-effect transistor; a nanopore sensor; electrochemiluminescence; a SMR sensor; a nanoelectrokinetic chip; and a microarray.

Smartphones may be used in combination with the devices of the present disclosure to detect a signal that indicates that target nucleic acids have been detected in the sample. In one embodiment, an RGB image is acquired every 30 seconds for the duration of 1 hour and the images are analyzed offline using software. In some embodiments, the RGB image is further demosaiced to a greyscale image. In some embodiments, the saturated pixels or pixels exhibiting two very different green submosaic values are excluded. In other embodiments, a rectangular image region-of interest (ROI) is drawn within a detection zone and the reporter signal in the ROI is determined by using the pixel values.

A lateral flow assay (LFA) or lateral flow strip may be used in the methods and devices of the present disclosure to detect the nucleic acid in the biological sample. LFA platforms may be adapted to incorporate Cas effector proteins as a target sequence recognition element. In some embodiments, a commercially available universal test strip, the HybriDetect-Universal Lateral Flow Assay Kit, may be used. This dipstick was originally designed for qualitative or even quantitative rapid testing of proteins, antibodies or gene amplicons, but has been adapted to function in several LFA based CRISPR sensing methods (Bai et al., 2019; Chang et al., 2019; Gootenberg et al., 2018; Kaminski et al., 2020; Mukama et al., 2020b; Sullivan et al., 2019; Tsou et al., 2019). This platform offers a Streptavidin line as well as an antibody line that can capture anti-FITC coated AuNPs. By dual labeling of single-stranded reporter nucleotides on both 5′ end and 3′ end with Biotin and FITC, the intensity of the test line can be a measure for the amount of collateral cleavage performed by Cas effector proteins. By calibration the collateral cleavage activity can be related to the target sequence concentration present in the original sample. It has been shown that in this way femtomolar (10-15 M) concentrations cand be measured within one hour without target sequence amplification. In some embodiments, isothermal amplification is performed prior to CRISPR chemistry detection to further lower to a LOD of attomolar (10-18 M) or even zeptomolar (10-21 M) concentrations.

### Y-Chromosome Detection

The disclosure provides methods, compositions, tests, and kits for detecting Y-chromosome nucleic acids in a biological sample obtained from a pregnant subject. In some embodiments, the Y-chromosome nucleic acids are cell-free fetal nucleic acids (e.g., cffDNA). In some embodiments, the Y-chromosome nucleic acids are genomic fetal nucleic acids from a fetal cell (e.g., gfDNA). In some embodiments, the Y-chromosome nucleic acids are placental sourced.

The disclosure provides compositions for detecting Y-chromosome nucleic acids in a biological sample. In some embodiments, the compositions are primers and/or probes that are capable of amplifying and detecting at least one target sequence on the Y-chromosome. The probe set may comprise one or more polynucleotide probes. Individual polynucleotide probes comprise a nucleotide sequence derived from the nucleotide sequence of the target sequences or complementary sequences thereof. The nucleotide sequence of the polynucleotide probe is designed such that it corresponds to, or is complementary to the target sequences. The polynucleotide probe can specifically hybridize under either stringent or lowered stringency hybridization conditions to a region of the target sequences. The selection of the polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the disclosure, the polynucleotide probe is complementary to a region of a target mRNA derived from a target sequence in the probe set. Computer programs can also be employed to select probe sequences that may not cross hybridize or may not hybridize non-specifically.

The polynucleotide target sequences of the disclosure may range in length from about 15 nucleotides to the full length of the target sequence on the Y-chromosome. In one embodiment of the disclosure, the polynucleotide target sequences are at least about 15 nucleotides in length. In another embodiment, the polynucleotide target sequences are at least about 20 nucleotides in length. In a further embodiment, the polynucleotide target sequences are at least about 25 nucleotides in length. In another embodiment, the polynucleotide target sequences are between about 15 nucleotides and about 500 nucleotides in length. In other embodiments, the polynucleotide target sequences are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the target sequences are at least 15 nucleotides in length. In some embodiments, the target sequences are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The present disclosure further provides primers and primer pairs capable of amplifying target sequences on the Y-chromosome. The nucleotide sequences of the primer set may be provided in computer-readable media for in silico applications and as a basis for the design of appropriate primers for amplification of one or more target sequences of the primer set.

Primers based on the nucleotide sequences of target sequences can be designed for use in amplification of the target sequences. In one embodiment, the primers or primer pairs, when used in an amplification reaction, specifically amplify at least a portion of a nucleic acid sequence of a target selected from the Y-chromosome. In some embodiments, the target sequences are present on the Y-chromosome in multiple locations. In certain embodiments, the target sequences are present in 2, 3, 4, 5, 6, 7, 8, 9, 10, about 15, about 20, about 25, about 50, about 75, about 100, about 150, about 200, about 300, about 400, about 500, about 750, about 1,000 locations on the Y-chromosome. The sensitivity of assays of the disclosure can be increased by detecting and/or amplifying target sequences that are present on the Y-chromosome in multiple locations. Multiple primer pairs can be used in the methods of the disclosure. For example, a duplex or multiplex amplification assay may be used to increase the detection limit of an assay of the disclosure. A label can be attached to or incorporated into a probe or primer polynucleotide to allow detection and/or quantitation of a target polynucleotide representing the target sequence of interest.

The analysis of a plurality of target sequences on the Y-chromosome may be carried out separately or simultaneously with one test sample. In some embodiments, the target on the Y-chromosome is SRY, DYS, and/or DAZ. An assay consisting of a combination of the target sequences referenced in the instant disclosure may be constructed. Such a panel may be constructed using 1, 2, 3, 4, 5, 6, 7, 8, 9, 10, 15, 20, or more or target sequences. The analysis of a single target sequence or subsets of target sequences comprising a larger panel of Y-chromosome targets could be carried out with the methods described within the instant disclosure to optimize assay sensitivity or specificity in various settings. The ratio of a target sequence on the Y-chromosome and a control sequence from an autosomal chromosome may be used in an algorithm of the disclosure for determining fetal sex.

The methods, compositions, and kits of the disclosure can be used to detect very small amounts of Y-chromosome DNA in a biological sample from a pregnant subject. In some embodiments, the methods of the disclosure can detect about 1 to 0.1 genomic equivalent of cffDNA in a sample, about 0.9 genomic equivalent of cffDNA in a sample, about 0.8 genomic equivalent of cffDNA in a sample, about 0.7 genomic equivalent of cffDNA in a sample, about 0.6 genomic equivalent of cffDNA in a sample, about 0.5 genomic equivalent of cffDNA in a sample, about 0.4 genomic equivalent of cffDNA in a sample, about 0.3 genomic equivalent of cffDNA in a sample, about 0.2 genomic equivalent of cffDNA in a sample, about 0.1 genomic equivalent of cffDNA in a sample. In other embodiments, the methods of the disclosure can detect a single copy of Y-chromosome DNA.

In other embodiments, the methods of the disclosure further comprise interpreting data generated when detecting the Y-chromosome DNA. In some embodiments, the interpreting is performed using a machine learning algorithm, a cycle-threshold (CT) algorithm, or artificial intelligence.

### Kits and Devices

Another aspect of the disclosure encompasses kits for collecting biological samples from pregnant subjects or for detecting Y-chromosome nucleic acids in a biological sample from the pregnant subject. A variety of kits having different components are contemplated by the disclosure. Generally speaking, the kit will include the means for detecting Y-chromosome in a biological sample from a pregnant subject. In another embodiment, the kit will include means for collecting a biological sample and instructions for use of the kit contents. In certain embodiments, the kit comprises a means for enriching or isolating fetal nucleic acids in a biological sample. In further aspects, the means for enriching or isolating fetal nucleic acids comprises reagents necessary to enrich or isolate fetal nucleic acids from a biological sample.

The kits of the disclosure may include instructions for decontaminating the site on the pregnant subject where the sample will be collected. In certain embodiments, the decontamination is performed by applying bleach to the site of collection, by applying an alcohol wipe to the site of collection, by treating the site of collection with ultra-violet light, by applying chlorhexidine gluconate, hydrogen peroxide, and/or iodine to the site of collection, by applying a brush (e.g., a nail brush) to the site of the collection.

The present disclosure further provides kits for obtaining a biological sample from a pregnant subject. The kits may comprise a blood collection tube, a lancet or a device useful for obtaining venous or capillary blood from the subject, a tourniquet, a bandage, an alcohol swab, a nail or skin brush, and instructions for using the kits. In some embodiments, the kits further comprise a decontaminating agent. In certain embodiments, the decontaminating agent is bleach, an alcohol wipe, chlorhexidine gluconate, hydrogen peroxide, and/or iodine. In other embodiments, the device for obtaining venous or capillary blood is a lancet (e.g., BD Microtainer contact-activated lancet), a syringe, and/or a push-button blood collection device (e.g., a TAP device). In some embodiments, the biological sample is collected into a tube, onto a card, and/or a swab.

Methods and kits of the disclosure can include instructions that provide a minimum gestational age or gestational age range for sample collection. In some embodiments, the minimum gestational age is 2 weeks, 3 weeks, 4 weeks, 5 weeks, 6 weeks, 7 weeks, 8 weeks, 9 weeks, 10 weeks, 11 weeks, 12 weeks, 13 weeks, 14, week, 15 weeks, 16 weeks, 17 weeks, 18 weeks, 19 weeks, 20 weeks, 21 weeks, 22 weeks, 23 weeks, 24 weeks, 25 weeks, 30 weeks, 35 weeks, or 40 weeks. In some embodiments, the gestational age is 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days 42 days, 49 days, 56 days, 63 days, 70 days, 77 days, 84 days, 100 days, 150 days, 200 days, or 250 days.

The present disclosure provides devices for detecting the presence of one or more target fetal nucleic acids in a biological sample, the device comprising: a lateral flow strip; a detection region on said lateral flow strip comprising a detectable particle or label: a fluid sample comprising a maternal biological sample comprising fetal nucleic acids; wherein said detection region provides a visual colorimetric signal indicating the presence of the target fetal nucleic acid in the fluid sample in less than two hours by capillary flow. In some embodiments, the devices of the present disclosure further comprise a CRISPR composition, a preservative composition, an amplification composition, and/or a protectant composition. Device of the present disclosure may further comprise a decontaminating agent. In some embodiments, the decontaminating agent is bleach, an alcohol wipe, chlorhexidine gluconate, hydrogen peroxide, and/or iodine.

These and other embodiments of the present disclosure will readily occur to those of ordinary skill in the art in view of the disclosure herein.

### EXAMPLES

The disclosure will be further understood by reference to the following examples, which are intended to be purely exemplary of the disclosure. These examples are provided solely to illustrate the claimed disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description. Such modifications are intended to fall within the scope of the appended claims.

### Example 1: Rapid Determination of Fetal Sex from Maternal Blood

Rapid determination of fetal sex from maternal blood is performed as follows. A blood sample of 250 ul is collected from a pregnant woman into a blood collection device containing amplification compositions including a biotin labeled primer, a FAM labeled probe, and an isothermal DNA polymerase enzyme. Isothermal amplification of target nucleic acids on the Y-chromosome are carried out by warming the sample at 39° C. for 20 minutes. About 5-10 ul of the sample is transferred to a tube containing 100 ul of flow strip buffer. A custom lateral flow strip that is designed to detect biotin and FAM labeled amplification products is placed in the solution in the tube. The solution flows up the lateral flow strip and a red band forms in a test zone on the strip within 5 to 15 minutes if the sample contains the target nucleic acid on the Y-chromosome. A positive test band indicated that the sex of the fetus is male. If no band is visible in the test zone after 5 to 15 minutes, then the target nucleic acid on the Y-chromosome is not present in the sample and the sex of the fetus is female. These results show that a fetal sex test of the present disclosure can accurately determine fetal sex in less than 60 minutes.

In another set of experiments, rapid determination of fetal sex from maternal blood is performed as follows. A blood sample of 250 ul is collected from a pregnant woman into a blood collection device containing a CRISPR composition including a CRISPR/Cas effector protein, a guide RNA, and a FAM-biotin labeled reporter DNA molecule. The sample and CRISPR composition are incubated at 37° C. for 45 minutes. CRISPR induced cleavage of the labeled reporter DNA molecule occurs only if Y-chromosome DNA is present in the sample. Next, about 10 ul of the sample is transferred to a tube containing 100 ul of flow strip buffer. A custom lateral flow strip that is designed to detect biotin and FAM labeled amplification products is placed in the solution in the tube. The solution flows up the lateral flow strip and a red band forms in a test zone on the strip within 5 to 15 minutes if the sample contains the target nucleic acid on the Y-chromosome. A positive test band indicates that the sex of the fetus is male. If no band is visible in the test zone after 5 to 15 minutes, then the target nucleic acid on the Y-chromosome is not present in the sample and the sex of the fetus is female. These results show that a fetal sex test of the present disclosure can accurately determine fetal sex in less than 60 minutes.

In another set of experiments, rapid determination of fetal sex from maternal blood is performed as follows. A blood sample of 250 ul is collected from a pregnant woman into a blood collection device containing amplification compositions including primers and an isothermal DNA polymerase enzyme and a CRISPR composition including a CRISPR/Cas effector protein, a guide RNA, and a FAM-biotin labeled reporter DNA molecule. Isothermal amplification of target nucleic acids on the Y-chromosome are carried out by warming the sample at 37° C. for 30 minutes. During amplification, CRISPR induced cleavage of the labeled reporter DNA molecule occurs only if Y-chromosome DNA is present in the sample. Next, about 10 ul of the sample is transferred to a tube containing 100 ul of flow strip buffer. A custom lateral flow strip that is designed to detect biotin and FAM labeled amplification products is placed in the solution in the tube. The solution flows up the lateral flow strip and a red band forms in a test zone on the strip within 5 to 15 minutes if the sample contains the target nucleic acid on the Y-chromosome. A positive test band indicated that the sex of the fetus is male. If no band is visible in the test zone after 5 to 15 minutes, then the target nucleic acid on the Y-chromosome is not present in the sample and the sex of the fetus is female. These results show that a fetal sex test of the present disclosure can accurately determine fetal sex in less than 60 minutes.

### Example 2: Rapid Determination of Fetal Sex from Maternal Blood at 8 Weeks Gestation Using Isothermal Amplification

Rapid determination of fetal sex from maternal blood was performed using isothermal amplification as follows. Venous blood was collected from pregnant women at approximately 8 to 9 weeks gestation. Blood samples were processed and tested approximately 24-72 hours after collection.

Three milliliters (3 ml) of maternal blood was collected from the arm of all participants using a 3 ml SneakPeek blood collection tube (Gateway Genomics, California). The blood samples were mailed to a clinical lab for processing. The maternal blood samples were centrifuged at 1,600 g for 15 minutes to separate plasma from whole blood. Next, cfDNA was isolated from 100 ul plasma samples using a MagMAX Cell-Free DNA Isolation Kit (ThermoFisher) according to the manufacturer's instructions.

Isolated cell-free DNA (5 ul) was dispensed into 96-well plates and reacted with Twist Exo Liquid Kit (TwistDx) reagents according to the manufacturer's recommendations for a final RPA reaction volume of 50 ul per well, including primers and a fluorescent labeled probe. Male cell-free DNA was detected using a multi-copy target sequence on the Y-Chromosome. Isothermal amplification of target nucleic acids on the Y-chromosome were carried out by warming the sample at 37° C. for 40 minutes. Fluorescence from each well was measured every 30 seconds for 40 minutes and the fluorescent signal was recorded on an amplification plot. Samples from pregnant women carrying male fetuses showed exponential elevation of fluorescence signal within 15 to 25 minutes. Samples from pregnant women carrying female fetuses did not show any significant alteration in the fluorescence intensity.

These results showed that methods and compositions of the disclosure are useful for rapid determination of fetal sex from maternal blood. These results further showed that the methods and compositions of the disclosure are useful for performing isothermal amplification of nucleic acids to determine the sex of a fetus in a maternal blood sample. These results showed that a fetal sex test of the present disclosure can accurately determine fetal sex in less than 60 minutes.

Various modifications of the disclosure, in addition to those shown and described herein, will become apparent to those skilled in the art from the foregoing description. Such modifications are intended to fall within the scope of the appended claims. All references cited herein are hereby incorporated by reference herein in their entirety.

What is claimed is:

1. A method for determining the sex of a fetus comprising: a) obtaining a biological sample from a subject who is pregnant or suspected of being pregnant comprising fetal nucleic acids; b) performing isothermal amplification on one or more target fetal nucleic acids in the sample to generate reporter molecules if the target nucleic acids are present in the sample; c) detecting the reporter molecules, wherein detection of the reporter molecules indicates the presence of target fetal nucleic acids in the sample; and d) determining the sex of the fetus based on detecting the presence of the target fetal nucleic acids in the sample.

2. A method comprising: a) obtaining a biological sample from a subject who is pregnant or suspected of being pregnant comprising fetal nucleic acids; b) performing isothermal amplification on one or more target fetal nucleic acids in the sample to generate reporter molecules if the target nucleic acids are present in the sample; and c) detecting the reporter molecules, wherein detection of the reporter molecules indicates the presence of target fetal nucleic acids in the sample.

3. A method for determining the sex of a fetus comprising: a) obtaining a biological sample from a subject who is pregnant or suspected of being pregnant comprising fetal nucleic acids; b) contacting the sample with a CRISPR/Cas effector protein, a guide RNA, and a labeled reporter DNA molecule, wherein the labeled reporter DNA molecule is cleaved if one or more target fetal nucleic acids are present in the sample; c) detecting a signal produced by the cleavage of the labeled reporter DNA molecule by the CRISPR/Cas effector protein, wherein detection of the signal indicates the presence of the target fetal nucleic acids in the sample; and d) determining the sex of the fetus based on detecting the presence of the target fetal nucleic acids in the sample.

4. A method comprising: a) obtaining a biological sample from a subject who is pregnant or suspected of being pregnant comprising fetal nucleic acids; b) contacting the sample with a CRISPR/Cas effector protein, a guide RNA, and a labeled reporter DNA molecule, wherein the labeled reporter DNA molecule is cleaved if one or more target fetal nucleic acids are present in the sample; and c) detecting a signal produced by the cleavage of the labeled reporter DNA molecule by the CRISPR/Cas effector protein, wherein detection of the signal indicates the presence of the target fetal nucleic acids in the sample.

5. A method for determining the sex of a fetus comprising: a) obtaining a biological sample from a subject who is pregnant or suspected of being pregnant comprising fetal nucleic acids; b) performing isothermal amplification on one or more target fetal nucleic acids in the sample; c) contacting the sample with a CRISPR/Cas effector protein, a guide RNA, and a labeled reporter DNA molecule, wherein the labeled reporter DNA molecule is cleaved if one or more target fetal nucleic acids are present in the sample; d) detecting a signal produced by the cleavage of the labeled reporter DNA molecule by the CRISPR/Cas effector protein, wherein detection of the signal indicates the presence of the target fetal nucleic acids in the sample; and e) determining the sex of the fetus based on detecting the presence of the target fetal nucleic acids in the sample.

6. A method comprising: a) obtaining a biological sample from a subject who is pregnant or suspected of being pregnant comprising fetal nucleic acids; b) performing isothermal amplification on one or more target fetal nucleic acids in the sample; c) contacting the sample with a CRISPR/Cas effector protein, a guide RNA, and a labeled reporter DNA molecule, wherein the labeled reporter DNA molecule is cleaved if one or more target fetal nucleic acids are present in the sample; and d) detecting a signal produced by the cleavage of the labeled reporter DNA molecule by the CRISPR/Cas effector protein, wherein detection of the signal indicates the presence of the target fetal nucleic acids in the sample.

7. The method of any one of claims 1 to 6, further comprising determining the sex of a fetus based on the detection of one or more target fetal nucleic acid sequences on the Y-chromosome.

8. The method of any one of claims 1 to 7, wherein the target fetal nucleic acid is cell-free fetal DNA.

9. The method of any one of claims 1 to 7, wherein the target nucleic acid is a single copy target sequence or a multicopy target sequence.

10. The method of claim 9, wherein the multicopy target sequence is present on the Y-chromosome in more than 20 locations, 30 locations, 40 locations, 50 locations, 100 locations, or 1,000 locations.

11. The method of any one of claims 1 to 10, wherein the sample is blood, plasma, serum, saliva, urine, and/or cervical mucus.

12. The method of any one of claims 1 to 11, wherein the detection of the reporter molecule is detected in less than 30 minutes.

13. The method of any one of claims 1 to 11, wherein the detection of the reporter molecule is detected in less than 60 minutes.

14. The method of any one of claims 1 to 13, wherein the detection is carried out with an Electrochemical chip, a Graphene, field-effect transistor, a nanopore sense, an SMR sensor, a nanoelectrokinetic chip, or microarray.

15. The method of any one of claims 1 to 14, further comprising amplifying the target DNA in the sample by loop-mediated isothermal amplification (LAMP), helicase-dependent amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR), and/or isothermal multiple displacement amplification (IMDA).

16. The method of any one of claims 1 to 15, further comprising contacting the biological sample with a preservative composition.

17. The method of any one of claims 1 to 16, further comprising contacting the biological sample with a protectant composition.

18. The method of claim 17, wherein the protectant composition comprises dextran, trehalose, and/or pullulan.

19. The method of any one of claims 3 to 18, wherein the guide RNA comprises more than one crRNA.

20. The method of any one of claims 3 to 19, wherein the CRISPR/Cas effector protein is Cas9, Cas12a, Cas 14a/b, and/or Cas 13a/b.

21. The method of claim any one of claims 16 to 20, wherein the preservative composition comprises an anti-coagulant (e.g., ethylenediaminetetraacetic acid (EDTA), ethylene glycol-bis(β-aminoethyl ether)-N,N,N′,N′-tetraacetic acid (EGTA), heparin), an antimicrobial (e.g., imidazolidinyl urea), a sugar, and/or an amino acid.

22. The method of any one of claims 1 to 21, further comprising enriching the sample for fetal nucleic acids.

23. The method of claim 22, wherein the enrichment is achieved by separating plasma from whole blood, by selectively capturing fetal nucleic acids from the biological sample, and/or by selectively degrading maternal nucleic acids in the biological sample.

24. The method of any one of claims 1 to 23, wherein the fetal nucleic acids are cell-free fetal nucleic acids or genomic fetal nucleic acids from a fetal cell.

25. The method of any one of claims 1 to 24, further comprising isolating, enriching, and/or concentrating the fetal nucleic acids.

26. The method of any one of claims 1 to 25, wherein the sex of the fetus is determined with at least 90% accuracy.

27. The method of any one of claims 1 to 26, wherein the gestational age of the fetus is between 4 weeks and 20 weeks.

28. The method of any one of claims 1 to 27, wherein the sample volume is less than 1 ml.

29. The method of any one of claims 14 to 28, wherein the preservative is an anti-coagulant (e.g., ethylenediaminetetraacetic acid (EDTA), ethylene glycol-bis(O-aminoethyl ether)-N,N,N′,N′-tetraacetic acid (EGTA), heparin), an antimicrobial (e.g., imidazolidinyl urea), a sugar, and/or an amino acid.

30. A device for detecting the presence of one or more target fetal nucleic acids in a biological sample, the device comprising: a lateral flow strip; a detection region on said lateral flow strip comprising a detectable particle or label: a fluid sample comprising a maternal biological sample comprising fetal nucleic acids; wherein said detection region provides a visual colorimetric signal indicating the presence of the target fetal nucleic acid in the fluid sample in less than two hours by capillary flow.

31. The device of claim 30, further comprising a CRISPR composition, a preservative composition, an amplification composition, and/or a protectant composition.
